(19) United States
(12) Patent Application Publication
(10) Pub. No.: US 2022/0008034 A1
(43) Pub. Date: Jan. 13, 2022

(54) ULTRASOUND DETECTION DEVICE

(71) Applicant: KYOCERA Corporation, Kyoto-shi, Kyoto (JP)

(72) Inventor: Yohei SATO, Osaka-shi, Osaka (JP)

(21) Appl. No.: 17/289,070

(22) PCT Filed: Oct. 29, 2019
(86) PCT No.: PCT/JP2019/042296

§ 371 Date: Apr. 27, 2021

(30) Foreign Application Priority Data
Oct. 31, 2018 (JP) 2018-205182

(51) Int. Cl. A61B 8/12; A61B 8/00; B06B 1/02

(52) U.S. Cl. CPC A61B 8/12; A61B 8/4444; B06B 2201/51; B06B 1/0215; A61B 8/4494

(57) ABSTRACT

An ultrasound detection device includes a probe and a transceiver. The probe includes first and second transducers, and an amplifier circuit. The first transducer transmits ultrasound. The second transducer includes a first electrode connected to a first wire, and converts the ultrasound into an electrical signal. The amplifier circuit includes first and second transistors. The first transistor includes a third electrode connected to the first wire, a fourth electrode as a gate or a base connected to a second electrode of the second transducer, and a fifth electrode connected to the fourth electrode via a resistor and connected to a second wire via a resistor. The second transistor includes an electrode connected to the first wire, an electrode as a gate or a base connected to the fifth electrode, and an electrode connected to the second wire via a resistor and connected to the second wire via a capacitor.

### CROSS-REFERENCE TO RELATED APPLICATION

The present application is a National Phase entry based on PCT Application No. PCT/JP2019/042296 filed on Oct. 29, 2019, entitled “ULTRASOUND DETECTION DEVICE”, which claims the benefit of Japanese Patent Application No. 2018-205182, filed on Oct. 31, 2018, entitled “ULTRASOUND DETECTION DEVICE”. The contents of which are incorporated by reference herein in their entirety.

### FIELD

Embodiments of the present disclosure relate generally to an ultrasound detection device.

### BACKGROUND

An ultrasound detection device capable of performing intravascular ultrasound (IVUS) enabling acquisition of a tomographic image in a blood vessel utilizing reflection of ultrasound has been known.

The ultrasound detection device typically has a configuration in which an ultrasound probe (a probe portion) and a pulser-receiver including a transceiver to transmit and receive signals are electrically connected by a cable inserted into a catheter. In the ultrasound detection device, the pulser-receiver applies a transmission signal for driving of a large voltage to a transducer of the probe portion via the cable for signals in the catheter, for example, so that the transducer generates ultrasound. In this case, the transducer receives ultrasound reflected by each portion of the blood vessel, and generates a weak reception signal. The weak reception signal is amplified by an amplifier circuit in the probe portion, and is then transmitted to the pulser-receiver via the cable for signals in the catheter. For application of a voltage to the transducer, a wire for signals (also referred to as a signal wire) is electrically connected to a first electrode of the transducer, and a wire to apply a potential to be the basis for a voltage (also referred to as a reference wire) is electrically connected to a second electrode of the transducer, for example. The reference wire includes a wire having been grounded (also referred to as a ground wire), for example.

As for such an ultrasound detection device, technology to provide a circuit (also referred to as a switch circuit) to block passage of the transmission signal and allow the weak reception signal to pass therethrough before the amplifier circuit in the probe portion has been proposed. This makes the amplifier circuit in the probe portion less likely to be broken by input of the transmission signal to generate a large voltage, for example. The switch circuit includes a diode bridge circuit or the like, for example.

### SUMMARY

An ultrasound detection device is disclosed.

In one embodiment, the ultrasound detection device includes a probe portion, a transceiver, a first wire, and a second wire. The probe portion is capable of transmitting and receiving ultrasound to and from an object. The transceiver is capable of transmitting and receiving a signal to and from the probe portion, and applying a power supply voltage to the probe portion. The first wire electrically connects the transceiver and the probe portion, and enables the transceiver to apply a reference potential to the probe portion. The second wire electrically connects the transceiver and the probe portion, and enables the transceiver to transmit a voltage signal to the probe portion and enables the probe portion to transmit a current signal to the transceiver. The probe portion includes a first transducer, a second transducer, a bidirectional diode, and an amplifier circuit. The first transducer is capable of transmitting ultrasound to the object in response to application of the voltage signal. The second transducer is capable of receiving ultrasound from the object, and converting the ultrasound into an electrical signal. The bidirectional diode connects the second wire and the first transducer. The amplifier circuit is capable of amplifying a current, and outputting a current signal to the second wire in response to the electrical signal. The second transducer includes a first electrode electrically connected to the first wire and a second electrode. The amplifier circuit includes a first transistor, a first resistive element, a second transistor, a second resistive element, and a first capacitive element. The first transistor includes a third electrode, a fourth electrode, and a fifth electrode, and is in a common source configuration or a common emitter configuration. The third electrode is connected to the first wire, and acts as a source or an emitter. The fourth electrode is electrically connected to the second electrode of the second transducer, and acts as a gate or a base. The fifth electrode is electrically connected to the fourth electrode via a resistive element, and acts as a drain or a collector. The first resistive element electrically connects the fifth electrode and the second wire. The second transistor includes a sixth electrode, a seventh electrode, and an eighth electrode, and is of a different conductivity type from the first transistor and is in a common drain configuration or a common collector configuration, or is of the same conductivity type as the first transistor and is in a common source configuration or a common emitter configuration. The sixth electrode is connected to the first wire. The seventh electrode is electrically connected to the fifth electrode, and acts as a gate or a base. The second resistive element electrically connects the eighth electrode and the second wire. The first capacitive element connects the eighth electrode and the second wire. The transceiver is capable of detecting a voltage in accordance with the current signal using an internal resistor.

### BRIEF DESCRIPTION OF THE DRAWINGS

FIG. 1 illustrates a diagram showing one example of a configuration of an ultrasound detection device according to a first embodiment.

FIG. 2A illustrates a diagram showing one example of a configuration including a tip portion of a catheter portion in a part IIa of FIG. 1. FIG. 2B illustrates a diagram showing one example of an imaginary cut surface portion of the catheter portion along a line IIb-IIb of FIG. 2A.

FIG. 3 illustrates a diagram showing one example of a circuit configuration of the ultrasound detection device according to the first embodiment.

FIG. 4 illustrates a diagram for explaining operation of an amplifier circuit according to the first embodiment.

FIG. 5 illustrates a diagram for explaining operation of the amplifier circuit according to the first embodiment.

FIG. 6 illustrates a diagram showing one example of a circuit configuration of an ultrasound detection device according to a second embodiment.

FIG. 7 illustrates a diagram showing one example of a circuit configuration of an ultrasound detection device according to a third embodiment.

FIG. 8 illustrates a diagram showing one example of a circuit configuration of an ultrasound detection device according to a fourth embodiment.

FIG. 9 illustrates a diagram showing a first example of a circuit configuration of an ultrasound detection device according to a fifth embodiment.

FIG. 10 illustrates a diagram showing a second example of the circuit configuration of the ultrasound detection device according to the fifth embodiment.

FIG. 11 illustrates a diagram showing a first example of a circuit configuration of an ultrasound detection device according to a sixth embodiment.

FIG. 12 illustrates a diagram showing a second example of the circuit configuration of the ultrasound detection device according to the sixth embodiment.

FIG. 13 illustrates a diagram showing a third example of the circuit configuration of the ultrasound detection device according to the sixth embodiment.

FIG. 14 illustrates a diagram showing a fourth example of the circuit configuration of the ultrasound detection device according to the sixth embodiment.

### DETAILED DESCRIPTION

An ultrasound detection device capable of performing intravascular ultrasound (IVUS) enabling real-time viewing of a tomographic image in a blood vessel through measurement of reflection of ultrasound has been known, for example.

The ultrasound detection device typically has a configuration in which a probe portion and a pulser-receiver including a transceiver to transmit and receive signals to and from the probe portion are electrically connected by a cable in a catheter. In the ultrasound detection device, a transducer in the probe portion generates ultrasound in response to application of a transmission signal for driving of a large voltage of approximately −100 V or +100 V by the pulser-receiver via the cable for signals in the catheter, for example. The transducer generates a weak reception signal in response to ultrasound reflected by each portion of the blood vessel. The weak reception signal is amplified by an amplifier circuit in the probe portion, and is then transmitted to the pulser-receiver via the cable for signals in the catheter. The S/N ratio of the reception signal is thereby improved. For application of a voltage to the transducer, the transducer includes a first electrode electrically connected to a signal wire and a second electrode electrically connected to a reference wire, such as a ground wire, to apply a potential to be the basis for a voltage, for example.

As for an ultrasound detection device having such a configuration, it is envisaged that a switch circuit, such as a diode bridge circuit, to block passage of the transmission signal and allow the weak reception signal to pass therethrough is provided before the amplifier circuit in the probe portion, for example. This makes the amplifier circuit in the probe portion less likely to be broken by input of the transmission signal to generate a large voltage, for example.

When the amplifier circuit and the switch circuit are present in the probe portion, it is envisaged that at least two power supply wires to apply a voltage to drive the amplifier circuit and the switch circuit are inserted into the catheter in addition to two wires including the signal wire and the reference wire. In this case, at least four wires are required to be inserted into the catheter to transmit and receive the transmission signal and the reception signal using the pulser-receiver and a single transducer, for example. The number of wires inserted into the catheter can increase with increasing number of elements and circuits including the transducer, the amplifier circuit, and the switch circuit in the probe portion, for example. If the diameter of the catheter is increased by the increase in number of wires, the diameter of a blood vessel into which the catheter can be inserted can be limited, for example.

Such a problem is common not only to ultrasound detection devices capable of performing IVUS but also to ultrasound detection devices in general used in applications where the number of wires between the transceiver and the probe portion is to be reduced.

To address the problem, the inventor of the present disclosure has created technology enabling reduction in number of wires between the transceiver and the probe portion in the ultrasound detection device.

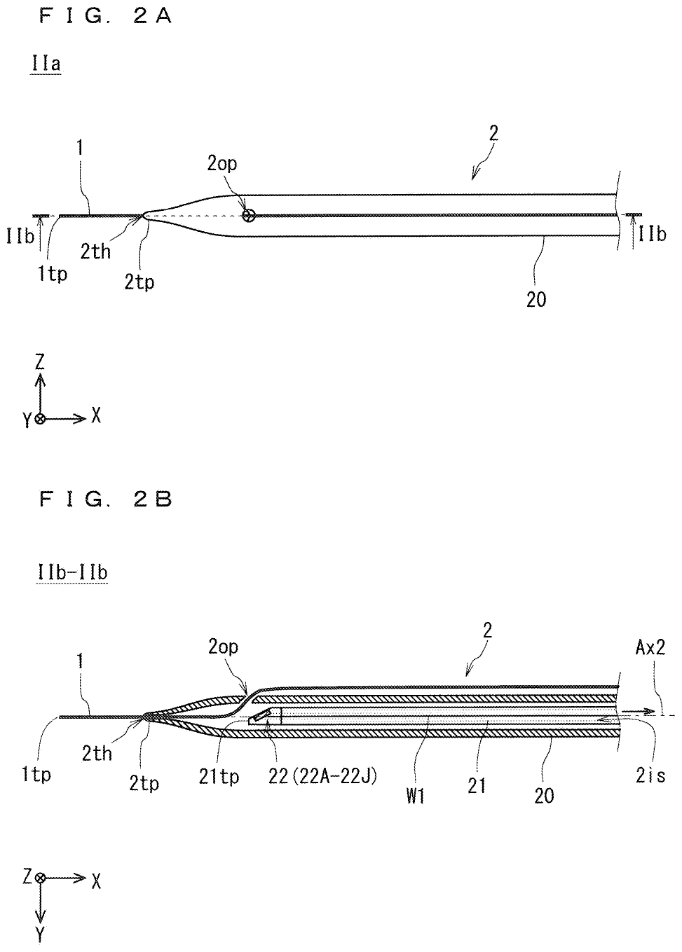

As for the foregoing, various embodiments will be described below with reference to the drawings. Components having similar configurations and functions bear the same reference signs in the drawings, and redundant description will be omitted below. The drawings are schematically shown. A right-handed XYZ coordinate system has been added to each of FIGS. 2A and 2B. In the XYZ coordinate system, a longitudinal direction of a catheter portion 2 toward a tip 2tp thereof is a −X direction, a direction along the diameter of the catheter portion 2 is a +Y direction, and a third direction orthogonal to each of a +X direction and the +Y direction is a +Z direction.

### 1. First Embodiment

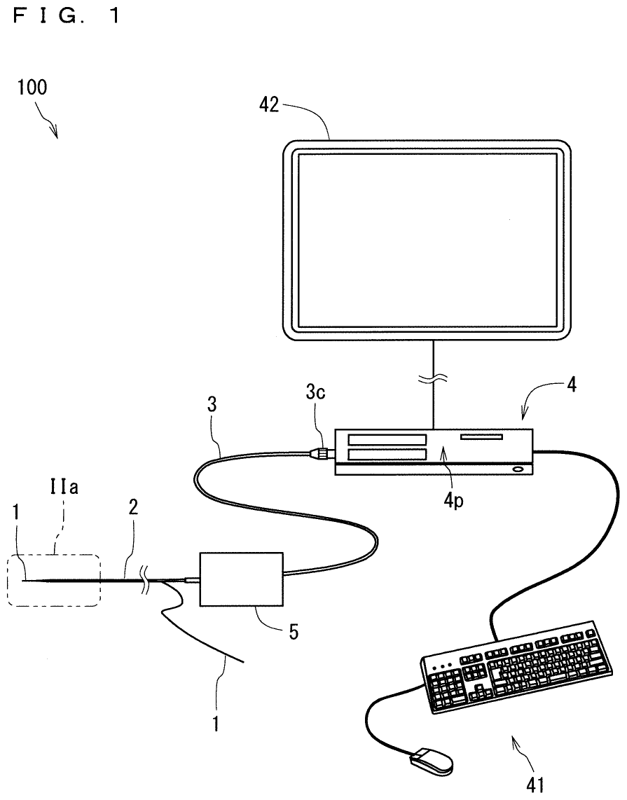

An ultrasound detection device 100 according to a first embodiment is an examination device including a catheter for a living body including a human body. The ultrasound detection device 100 according to the first embodiment will be described with reference to FIGS. 1 to 5.

### 1-1. Schematic Configuration of Ultrasound Detection Device

As shown in FIG. 1, the ultrasound detection device 100 includes a guide wire 1, the catheter portion 2, a cable portion 3, a body control unit 4, and a drive mechanism 5, for example.

The guide wire 1 is a linear member to guide the catheter portion 2 to a desired location in a meandering and curved lumen of a tubular body as a processing object of the living body. The tubular body can herein include a meandering and curved blood vessel, for example. Such a blood vessel can include heart coronary arteries, brain blood vessels, leg blood vessels and the like, for example. When the tubular body is the blood vessel, the lumen is a lumen of the blood vessel.

The catheter portion 2 is a thin tubular medical instrument with which various types of processing can be performed on the tubular body as the object. As shown in FIGS. 2A and 2B, the catheter portion 2 includes an elongated tubular body 20 and an elongated sensor portion 21 located in an internal space (also referred to as a lumen) 2is of the tubular body 20, for example.

The tubular body 20 has, at the tip 2tp thereof, a hole 2th through which the guide wire 1 has been inserted into the lumen 2is of the tubular body 20 relative to a tip 1tp of the guide wire 1. The tubular body 20 also has a hole 2op through which the guide wire 1 has been drawn out of the lumen 2is. The tubular body 20 can thus be slid along the guide wire 1. The guide wire 1 can thereby be inserted into the blood vessel, and the tubular body 20 can be inserted into the blood vessel along the guide wire 1, for example. The sensor portion 21 can be allowed to reach a target location, such as a lesion, along the tubular body 20, for example.

The sensor portion 21 is movable in the lumen 2is of the tubular body 20 in a longitudinal direction of the tubular body 20 along the tubular body 20, for example. The sensor portion 21 includes an ultrasound probe (also referred to as a probe portion) 22 located in the vicinity of a tip 21tp of the sensor portion 21 and a wire portion W1, for example. The probe portion 22 can transmit and receive ultrasound to and from the tubular body as the object, for example. Specifically, the probe portion 22 can transmit ultrasound in a direction crossing an axis that is imaginary (also referred to as an imaginary axis) Ax2 along a longitudinal direction of the sensor portion 21 in response to an electrical signal input from the body control unit 4 via the cable potion 3 and the wire portion W1, for example. The probe portion 22 can thus perform operation (also referred to as ultrasound transmission) to transmit ultrasound to the tubular body as the object, for example. The probe portion 22 can also receive ultrasound, and convert the ultrasound into an electrical signal, for example. The probe portion 22 can amplify the electrical signal, and then transmit the amplified electrical signal to the body control unit 4 via the wire portion W1.

The cable portion 3 is connected to the catheter portion 2 at a first end in a longitudinal direction thereof, for example. The cable portion 3 includes, at a second end in the longitudinal direction thereof, a connector 3c removably connected to the body control unit 4, for example. The body control unit 4 can thus transmit and receive various signals to and from the catheter portion 2 via the cable portion 3, for example. The body control unit 4 may supply power to the catheter portion 2 via the cable portion 3, for example.

The drive mechanism 5 can mechanically rotate the sensor portion 21 located in the lumen 2is of the tubular body 20 about the imaginary axis Ax2 along the longitudinal direction of the sensor portion 21, for example. The probe portion 22 can thus acquire an electrical signal pertaining to a cross-sectional structure at one location in the longitudinal direction of the tubular body as the object per rotation about the imaginary axis Ax2, for example. Such a scheme in which the probe portion 22 mechanically rotates is referred to as a mechanical scheme.

The body control unit 4 can control operation of each portion of the ultrasound detection device 100, for example. The body control unit 4 includes an input unit 41, an output unit 42, a transceiver 4p and the like, for example.

The input unit 41 can input a signal in accordance with operation of a user who uses the body control unit 4, for example. The input unit 41 can include an operating unit, a microphone, various sensors and the like, for example. The operating unit can include a mouse and a keyboard capable of inputting a signal in accordance with operation of the user. The microphone can input a signal in accordance with a voice of the user. The various sensors can input a signal in accordance with movement of the user.

The output unit 42 can output various information pieces, for example. The output unit 42 can include a display, a speaker and the like, for example. The display can visibly output the various information pieces so that the user can recognize the information pieces, for example. The display may herein be in the form of a touch panel integrated with at least part of the input unit 41. The speaker can audibly output the various information pieces so that the user can recognize the information pieces, for example. The various information pieces output from the output unit 42 can include image information pertaining to the cross-sectional structure of the tubular body as the object acquired using the sensor portion 21, for example.

The transceiver 4p can transmit and receive an electrical signal to and from the probe portion 22 and apply a power supply voltage to the probe portion 22, for example. The electrical signal transmitted and received between the transceiver 4p and the probe portion 22 can include a voltage signal and a current signal, for example. The transceiver 4p is also referred to as a pulser-receiver, for example. The transceiver 4p includes an internal resistor, and can detect a voltage in accordance with the current signal received from the probe portion 22 using the internal resistor. The electrical resistance of the internal resistor is set to have a predetermined value of approximately 50Ω, for example. The transceiver 4p may include an amplifier to amplify the current signal, and then detect the voltage in accordance with the amplified current signal using the internal resistor or an amplifier to amplify a voltage generated by the internal resistor in accordance with the current signal, and then detect the amplified voltage.

### 1-2. Circuit Configuration of Ultrasound Detection Device

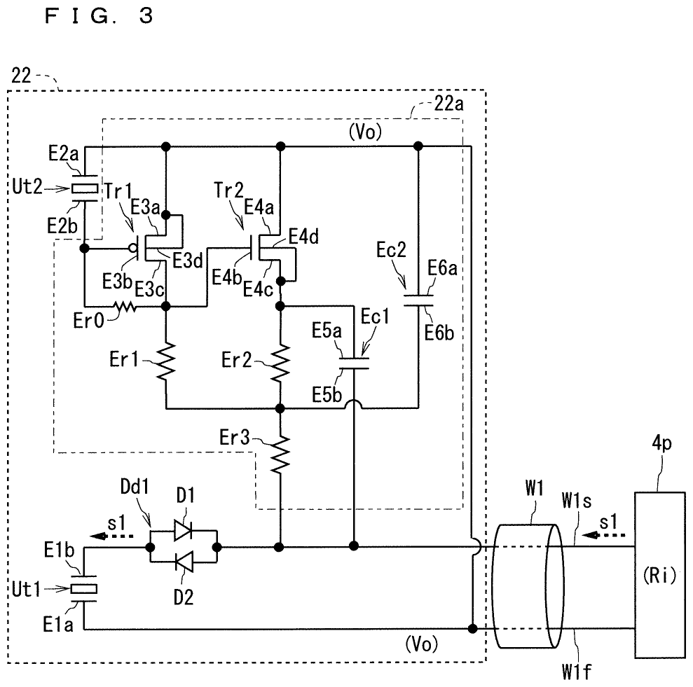

As shown in FIG. 3, the wire portion W1 includes a first wire W1f and a second wire W1s.

The first wire W1f electrically connects the transceiver 4p and the probe portion 22, for example. The first wire W1f enables the transceiver 4p to apply a potential to be the basis (also referred to as a reference potential) Vo to the probe portion 22, for example. In the first embodiment, the reference potential Vo is set, for example, to +10 V or the like as a predetermined positive potential. The reference potential Vo may be a predetermined positive potential of approximately +1 V to +30 V, for example. From another perspective, the first wire W1f has a function as a wire to supply power (also referred to as a power supply wire) to the probe portion 22, for example.

The second wire W1s electrically connects the transceiver 4p and the probe portion 22, for example. The second wire W1s enables the transceiver 4p to transmit an electrical signal to the probe portion 22, and enables the probe portion 22 to transmit an electrical signal to the transceiver 4p, for example. In the first embodiment, the electrical signal transmitted from the transceiver 4p to the probe portion 22 via the second wire W1s is the voltage signal, and the electrical signal transmitted from the probe portion 22 to the transceiver 4p via the second wire W1s is the current signal.

As shown in FIG. 3, the probe portion 22 includes a first transducer Ut1, a second transducer Ut2, a bidirectional diode Dd1, and an amplifier circuit 22a, for example.

<1-2-1. First Transducer>

The first transducer Ut1 can perform ultrasound transmission to transmit ultrasound to the tubular body as the object in response to application of the voltage signal as the electrical signal from the transceiver 4p to the probe portion 22, for example. The first transducer Ut1 may be a piezoelectric element or the like, for example. In the first embodiment, the first transducer Ut1 includes a 1A electrode E1a and a 1B electrode E1b, for example. The 1A electrode E1a is electrically connected to the first wire W1f, for example. The 1B electrode E1b is electrically connected to the second wire W1s via the bidirectional diode Dd1, for example.

<1-2-2. Second Transducer>

The second transducer Ut2 can receive ultrasound from the tubular body as the object, and convert the ultrasound into an electrical signal, for example. The second transducer Ut2 may be a piezoelectric element or the like, for example. In the first embodiment, the second transducer Ut2 includes a 2A electrode E2a as a first electrode and a 2B electrode E2b as a second electrode, for example. The 2A electrode E2a is electrically connected to the first wire W1f, for example.

<1-2-3. Bidirectional Diode>

The bidirectional diode Dd1 is electrically connected to the second wire W1s, for example. The bidirectional diode Dd1 allows the voltage signal as the electrical signal to pass therethrough toward the first transducer Ut1, for example. The bidirectional diode Dd1 does not allow an electrical signal having a strength less than a threshold to pass therethrough, for example. The bidirectional diode Dd1 includes a first diode D1 and a second diode D2 electrically connected in parallel with each other, for example. The first diode D1 allows a current to flow in a first direction. The second diode D2 allows a current to flow in a second direction opposite the first direction. The bidirectional diode Dd1 having such a configuration and function is also referred to as a TR switch.

In the first embodiment, the first direction is a direction from the first transducer Ut1 toward the transceiver 4p. The second direction is a direction from the transceiver 4p toward the first transducer Ut1. A signal exhibiting a negative potential can thus pass through the first diode D1 from the transceiver 4p toward the first transducer Ut1 in the first embodiment, for example. A voltage signal s1 exhibiting a negative minimum potential (also referred to as a minimum potential) Vmin having a larger absolute value than the positive reference potential Vo applied to the first wire W1f can herein pass through the first diode D1 from the transceiver 4p, and be applied to the 1B electrode E1b of the first transducer Ut1, for example. The voltage signal s1 may be a spike pulse exhibiting the negative minimum potential Vmin having a large absolute value, for example. The minimum potential Vmin of the voltage signal s1 is set to −100 V or the like, for example.

The first transducer Ut1 can herein generate ultrasound in response to the spike pulse having passed through the bidirectional diode Dd1, for example. The first transducer Ut1 can also receive ultrasound from the tubular body as the object, and convert the ultrasound into an electrical signal, for example. In this case, if the electrical signal generated by the first transducer Ut1 has a strength less than the threshold of the bidirectional diode Dd1, the electrical signal generated by the first transducer Ut1 is shielded by the bidirectional diode Dd1, and does not reach the second wire W1s. The threshold can include a voltage of the electrical signal, for example.

<1-2-4. Amplifier Circuit>

The amplifier circuit 22a can amplify a current, and output a current signal to the second wire W1s in response to the electrical signal, for example. In other words, the amplifier circuit 22a has a function of a so-called transconductance amplifier.

As shown in FIG. 3, the amplifier circuit 22a includes a first transistor Tr1, a first resistive element Er1, a second transistor Tr2, a second resistive element Er2, and a first capacitive element Ec1, for example. In the first embodiment, the amplifier circuit 22a includes a third resistive element Er3 and a second capacitive element Ec2, for example.

The first transistor Tr1 includes a 3A electrode E3a as a third electrode, a 3B electrode E3b as a fourth electrode, and a 3C electrode E3c as a fifth electrode, for example. The 3A electrode E3a is electrically connected to the first wire W1f, for example. The 3B electrode E3b is electrically connected to the 2B electrode E2b of the second transducer Ut2, for example. The 3C electrode E3c is electrically connected to the 3B electrode E3b via a resistive element Er0 to form a diode connection, for example. The resistive element Er0 has an electrical resistance of 10 kΩ or the like, for example. In the first embodiment, the first transistor Tr1 is a transistor having a metal oxide semiconductor (MOS) structure capable of forming a p-type channel having holes as majority carriers (also referred to as a PMOS transistor). In other words, the first transistor Tr1 is a transistor of a first conductivity type having holes as majority carriers. The 3A electrode E3a herein acts as a source electrode, for example. The 3B electrode E3b acts as a gate electrode, for example. The 3C electrode E3c acts as a drain electrode, for example. In this case, the first transistor Tr1 is a MOS transistor (also referred to as a first MOS transistor) in a common source configuration. In the first embodiment, the first transistor Tr1 includes a 3D electrode E3d as a back gate electrode electrically connected to the 3A electrode E3a, for example.

The first resistive element Er1 electrically connects the 3C electrode E3c of the first transistor Tr1 and the second wire W1s, for example. In other words, the 3C electrode E3c is electrically connected to the second wire W1s via the first resistive element Er1. In the first embodiment, the 3C electrode E3c is electrically connected to the second wire W1s via the first resistive element Er1 and the third resistive element Er3 in the stated order. The first resistive element Er1 is set to have an electrical resistance R1 of approximately 1000Ω, for example. The first resistive element Er1 may be a diffusion resistor or a well resistor less likely to be broken by application of a high voltage (also referred to as having a high breakdown voltage), a resistor of polysilicon formed on a dielectric film having a sufficient thickness or the like, for example.

The second transistor Tr2 includes a 4A electrode E4a as a sixth electrode, a 4B electrode E4b as a seventh electrode, and a 4C electrode E4c as an eighth electrode, for example. The 4A electrode E4a is electrically connected to the first wire W1f, for example. The 4B electrode E4b is electrically connected to the 3C electrode E3c of the first transistor Tr1, for example. In the first embodiment, the second transistor Tr2 is a transistor having a MOS structure capable of forming an n-type channel having electrons as majority carriers (also referred to as an NMOS transistor). In other words, the second transistor Tr2 is a transistor of a second conductivity type having electrons as majority carriers. The 4A electrode E4a can herein act as a drain electrode, for example. The 4B electrode E4b can act as a gate electrode, for example. The 4C electrode E4c can act as a source electrode, for example. In this case, the second transistor Tr2 is a MOS transistor (second MOS transistor) in a common drain configuration. In the first embodiment, the second transistor Tr2 includes a 4D electrode E4d as a back gate electrode electrically connected to the 4C electrode E4c, for example.

If the first transistor Tr1 and the second transistor Tr2 are the MOS transistors, for example, a portion of the amplifier circuit 22a including more elements can be implemented on a single semiconductor chip with miniaturization technology.

The second resistive element Er2 electrically connects the 4C electrode E4c of the second transistor Tr2 and the second wire W1s, for example. In other words, the 4C electrode E4c is electrically connected to the second wire W1s via the second resistive element Er2. In the first embodiment, the 4C electrode E4c is electrically connected to the second wire W1s via the second resistive element Er2 and the third resistive element Er3 in the stated order. The second resistive element Er2 can be set to have an electrical resistance R2 having a smaller value than the electrical resistance R1 of the first resistive element Er1, for example. In this case, if the electrical resistance R1 of the first resistive element Er1 is approximately 1000Ω, the electrical resistance R2 of the second resistive element Er2 can be set to approximately 500Ω, for example. As with the first resistive element Er1, the second resistive element Er2 may be a diffusion resistor or a well resistor having a high breakdown voltage, a resistor of polysilicon formed on a dielectric film having a sufficient thickness or the like, for example. The third resistive element Er3 is set to have an electrical resistance R3 having a small value of approximately 50Ω, for example.

The first capacitive element Ec1 connects the 4C electrode E4c of the second transistor Tr2 and the second wire W1s, for example. In other words, the 4C electrode E4c is connected to the second wire W1s via the first capacitive element Ec1. In the first embodiment, the first capacitive element Ec1 includes a 5A electrode E5a as a ninth electrode and a 5B electrode E5b as a tenth electrode, for example. The 5A electrode E5a is electrically connected to the 4C electrode E4c of the second transistor Tr2, for example. The 5B electrode E5b is electrically connected to the second wire W1s, for example. The first capacitive element Ec1 is set to have a capacitance C1 of approximately 75 pF, for example.

Herein, there are different routes diverging from the 4C electrode E4c, that is, a path along which a current can flow via the second resistive element Er2 and the third resistive element Er3 to the second wire W1s and a path along which a current can flow via the first capacitive element Ec1 to the second wire W1s, for example.

The second capacitive element Ec2 includes a 6A electrode E6a as an eleventh electrode and a 6B electrode E6b as a twelfth electrode, for example. The 6A electrode E6a is electrically connected to the first wire W1f, for example. The 6B electrode E6b is electrically connected to the second wire W1s via the third resistive element Er3, for example. The second capacitive element Ec2 can be set to have a capacitance C2 having a large value enabling formation on a single semiconductor chip, which is so-called on-chip formation, for example. In the first embodiment, the 6B electrode E6b is electrically connected to the second wire W1s via the third resistive element Er3, for example. The 6B electrode E6b is electrically connected to each of the resistive element Er0, the 3C electrode E3c, and the 4B electrode E4b via the first resistive element Er1. The 6B electrode E6b is further electrically connected to each of the 4C electrode E4c and the 5A electrode E5a via the second resistive element Er2. The third resistive element Er3 and the second capacitive element Ec2 can herein act as a low-pass filter to cut a high-frequency component of the voltage signal s1 input from the transceiver 4p into the amplifier circuit 22a via the second wire W1s for application to the first transistor Tr1 and the second transistor Tr2, for example. A change in voltage applied to the amplifier circuit 22a can thereby be slowed, for example. As a result, a high breakdown voltage of the amplifier circuit 22a for application of the voltage signal s1 can be improved, for example.

### 1-3. Current Amplification in Amplifier Circuit

<1-3-1. Reference State of Second Transducer Not Receiving Ultrasound>

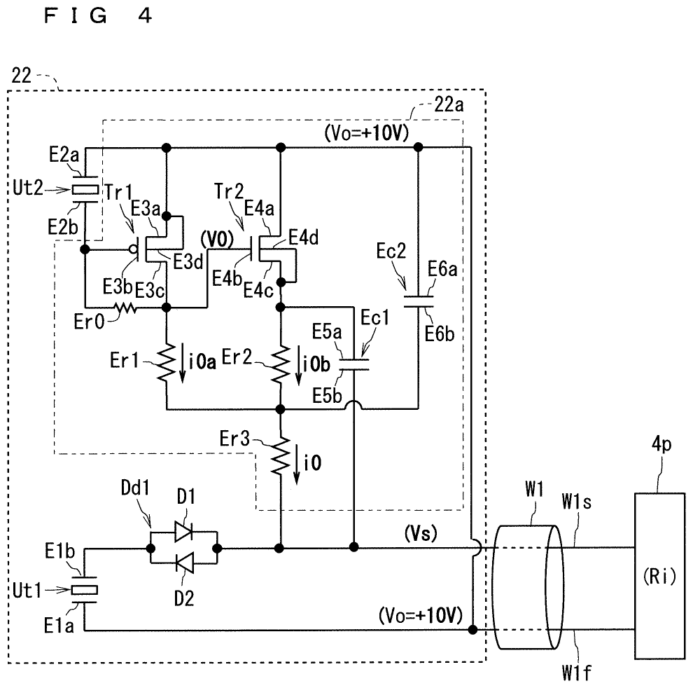

First, as shown in FIG. 4, a state of the transceiver 4p applying the constant reference potential Vo (e.g., +10 V) to the probe portion 22 via the first wire W1f and not applying the voltage signal s1 to the probe portion 22 via the second wire W1s is referred to as a reference state, for example. In the reference state, the voltage Vo (e.g., +10 V) is applied across the first wire W1f and a portion where the transceiver 4p is grounded (also referred to as a ground portion), for example. In this case, a steady-state current flows from the first wire W1f to the ground portion via the amplifier circuit 22a, the second wire W1s, and the internal resistor of the transceiver 4p. When the steady-state current flowing through the second wire W1s is i0, the steady-state current i0 herein exhibits a value obtained by dividing the voltage Vo by a resistance value (also referred to as a second combined resistance value) Rt2 of the sum of an electrical resistance (also referred to as a first combined resistance) Rt1 of the amplifier circuit 22a as a whole and an electrical resistance Ri (e.g., 50Ω) of the internal resistor of the transceiver 4p. In other words, the steady-state current i0 is expressed by an equation i0=Vo/Rt2.

The electrical resistance R2 (e.g., 500Ω) of the second resistive element Er2 herein weighs more heavily than the electrical resistance R1 (e.g., 1000Ω) of the first resistive element Er1 in the first combined resistance Rt1 of the amplifier circuit 22a . The second combined resistance Rt2 exhibits a value approximating to a value of the sum of the electrical resistance R2 and the electrical resistance Ri, for example. In other words, a relationship [second combined resistance Rt2]≈[electrical resistance R2]+[electrical resistance Ri] holds true. If the electrical resistance R2 is 500Ω, and the electrical resistance Ri is 50Ω, for example, the second combined resistance Rt2 is herein approximately 550Ω. In this case, the steady-state current i0 is approximately 18 mA (≈10 V/550Ω). The second wire W1s has a potential Vs of approximately 900 mV (≈50Ω×18 mA).

In the reference state, the 3B electrode E3b and the 3C electrode E3c of the first transistor Tr1 are short-circuited by the diode connection, for example. The 3B electrode E3b thus has a lower potential than the 3A electrode E3a. A voltage Vgs across the 3A electrode E3a as the source electrode and the 3B electrode E3b as the gate electrode thus exhibits a negative value. In the first transistor Tr1, a steady-state direct current i0a flows between the 3A electrode E3a as the source electrode and the 3C electrode E3c as the drain electrode. In this case, the voltage Vgs of the first transistor Tr1 exhibits a value in accordance with the direct current i0a. The voltage Vgs of the first transistor Tr1 herein has a value in accordance with a current I between the source and the drain as shown by Equation 1 and Equation 2.

Vgs=√{(2×I)/β}+Vth (Equation 1)

β=μ×Cox×(W/L) (Equation 2)

In Equation 1 and Equation 2, Vth is a threshold voltage of the first transistor Tr1. μ is mobility. Cox is a capacitance per unit area of a gate oxide film (also referred to as a unit gate oxide film capacitance). The unit gate oxide film capacitance Cox is expressed by an equation Cox=εox/tox where εox is the dielectric constant of an oxide contained in the gate oxide film, and tox is the thickness of the gate oxide film. W is a channel width. L is a channel length.

In the reference state, a positive potential V0 is applied to the 4B electrode E4b as the gate electrode of the second transistor Tr2 connected to the 3C electrode E3c when the steady-state direct current i0a flows in the first transistor Tr1. In the second transistor Tr2, a steady-state direct current i0b thus flows between the 4A electrode E4a as the drain electrode and the 4C electrode E4c as the source electrode. The direct current i0 (=i0a+i0b) thus steadily flows in the amplifier circuit 22a in the reference state.

<1-3-2. State of Second Transducer Receiving Ultrasound>

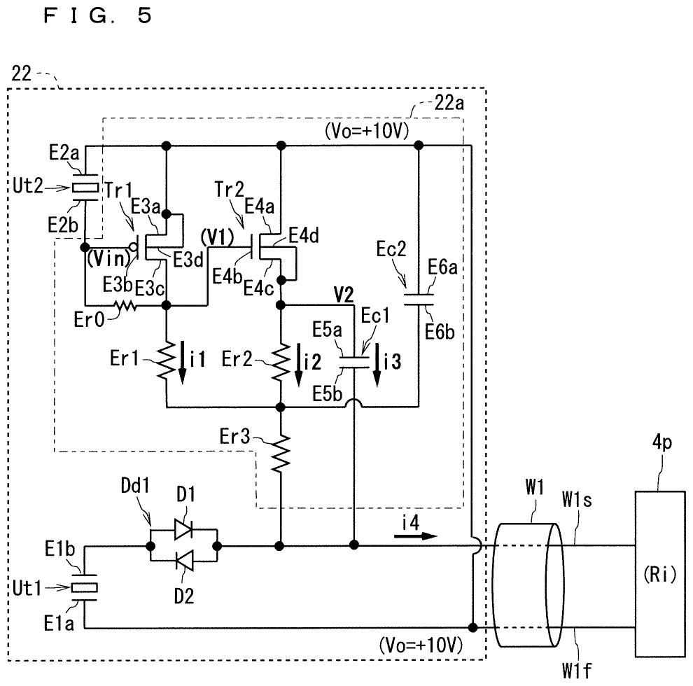

Assume herein that the second transducer Ut2 receives ultrasound, and converts the ultrasound into an electrical signal to apply a potential Vin to the 3B electrode E3b as the gate electrode of the first transistor Tr1 as shown in FIG. 5, for example. In this case, a current i1 flowing between the 3A electrode E3a as the source electrode and the 3C electrode E3c as the drain electrode is generated in the first transistor Tr1 in accordance with application of the potential Vin to the 3B electrode E3b. The current i1 is expressed by Equation 3.

i1=gm1×Vin (Equation 3)

In Equation 3, gm1 is transconductance (also referred to as mutual conductance) pertaining to the first transistor Tr1. The mutual conductance gm1 is set to approximately 30 mS, for example.

In this case, a potential V1 is applied to the 4B electrode E4b as the gate electrode of the second transistor Tr2 in accordance with input of the potential Vin into the first transistor Tr1. The potential V1 is expressed by Equation 4.

V1=gm1×Vin×R1 (Equation 4)

In Equation 4, gm1 is the transconductance (also referred to as the mutual conductance) pertaining to the first transistor Tr1. R1 is the electrical resistance of the first resistive element Er1. In view of a resistance (also referred to as an output resistance) Ro1 to output of the potential V1, for example, a voltage gain Av1 produced by amplification of the voltage Vin into the voltage V1 may herein be expressed by an equation Av1=gm1×{1/(1/R1+1/Ro1)}. In this case, bandwidth (also simply referred to as a band) ωc1 of a signal strength enabling amplification of the voltage Vin into the voltage V1 can be expressed by an equation ωc1=1/[{1/(1/R1+1/Ro1)}×Cg2] where Cg2 is a gate capacitance of the second transistor Tr2. Herein, the mutual conductance gm1 is set to approximately 30 mS, and the output resistance Ro1 is set to approximately 700Ω.

A current i2 flowing between the 4A electrode E4a as the drain electrode and the 4C electrode E4c as the source electrode is generated in the second transistor Tr2 in accordance with application of the potential V1 to the 4B electrode E4b. The second transistor Tr2 and the second resistive element Er2 herein form a common source (also referred to as source follower) circuit, for example. The potential V1 input into the 4B electrode E4b as the gate electrode and a potential V2 output from the 4C electrode E4c as the source electrode are thus substantially equal to each other in the second transistor Tr2. The potential V2 is expressed by Equation 5. The current i2 is expressed by Equation 6. A voltage gain Av2 produced by amplification of the voltage V1 into the voltage V2 may herein be expressed by an equation Av2=(gm2×Ro2)/{1+(gm2×Ro2)} where gm2 is mutual conductance, and Ro2 is an output resistance of the potential V2 in the second transistor Tr2, for example. In this case, a band ωc2 of a signal strength enabling amplification of the voltage V1 into the voltage V2 can be expressed by an equation ωc2=gm2/C2. C2 is the capacitance of the second capacitive element Ec2 as described above. Herein, the mutual conductance gm2 is set to approximately 133 mS, and the output resistance Ro2 is set to approximately 680Ω, for example. In this case, the voltage gain Av2 is approximately one.

V2≈V1=gm1×Vin×R1 (Equation 5)

i2=V2/R2≈gm1×Vin×(R1/R2) (Equation 6)

From Equation 3 and Equation 6, the current i2 and the current i1 have a relationship expressed by Equation 7.

i2=V2/R2≈gm1×Vin×(R1/R2)=(R1/R2)×i1 (Equation 7)

If the electrical resistance R1 of the first resistive element Er1 is 1000Ω, and the electrical resistance R2 of the second resistive element Er2 is 500Ω, for example, a relationship i2=2×i1 herein holds true.

In this case, the potential V2 is applied to the 5A electrode E5a and a potential of 0 V is applied to the 5B electrode E5b in the first capacitive element Ec1 to generate a current i3 as an alternating current component flowing through the first capacitive element Ec1. The current i3 is expressed by Equation 8.

i3=V2×jωC1 (Equation 8)

In Equation 8, jωC is admittance of the first capacitive element Ec1. More specifically, j is an imaginary unit. ω is an angular frequency. ω is equivalent to 2πf. f is a frequency of an alternating current. C1 is the capacitance of the first capacitive element Ec1.

The current i3 is expressed by Equation 9 by substituting Equation 5 for V2 in Equation 8, and further substituting Equation 3.

i3=V2×jωC1=gm1×Vin×(R1×jωC1)=(R1×jωC1)×i1 (Equation 9)

If the electrical resistance R1 is 1000Ω, the capacitance C1 is 75 pF, and the frequency f of the applied alternating voltage Vin is 60 MHz, for example, a relationship i3≈28.2×i1 herein holds true. In this case, the current i3 and the current i1 have a relationship in which the current i3 is obtained by amplifying the current i1 by a factor of approximately 28.2.

A current signal of a current (also referred to as a signal current) i4 as the sum of the current i1, the current i2, and the current i3 flows from the amplifier circuit 22a to the transceiver 4p via the second wire W1s. The signal current i4 output from the amplifier circuit 22a can herein be increased by setting the electrical resistance R1, the electrical resistance R2, and the capacitance C1 to appropriate large values, for example. For example, the signal current i4 is approximately 30.2 times the current i1 in the above-mentioned example. The amplifier circuit 22a can thus output the signal current i4 obtained by amplifying the current i1 by a factor of approximately 30, for example. A voltage in accordance with the current signal generated by the internal resistor of the transceiver 4p is thereby amplified, for example.

In this case, the transceiver 4p can detect, relative to a voltage corresponding to the steady-state direct current i0, a change in voltage in response to an increase and a decrease of the signal current i4 flowing in accordance with the potential Vin in response to reception of ultrasound by the second transducer Ut2, for example. The transceiver 4p can thus acquire the information pertaining to the cross-sectional structure of the tubular body as the object, for example.

As described above, in the ultrasound detection device 100, there are no dedicated wires for grounding and no power supply wires for driving of the switch circuit between the transceiver 4p and the probe portion 22, and there are only two wires including the first wire W1f and the second wire W1s therebetween, for example. Even in such a configuration, the first transducer Ut1 can transmit ultrasound in response to application of the voltage signal, the second transducer Ut2 can output the electrical signal in response to reception of ultrasound, and the amplifier circuit 22a can output the current signal amplified in response to the electrical signal. The number of wires between the transceiver 4p and the probe portion 22 can thereby be reduced in the ultrasound detection device 100, for example.

### 1-4. Other Properties of Amplifier Circuit

<1-4-1. High Breakdown Voltage and High Mutual Conductance of Amplifier Circuit>

If the transistors of the amplifier circuit are the MOS transistors less likely to be broken by application of a high voltage (having a high breakdown voltage), for example, it is envisaged that the gate oxide film is caused to have a significant thickness. An increase in thickness of the gate oxide film, however, can reduce the gain and the band of the signal strength enabling amplification. There is room for improvement in terms of a high breakdown voltage and high mutual conductance of the amplifier circuit, for example.

In the amplifier circuit 22a according to the first embodiment, a voltage as a difference between the reference potential Vo and the minimum potential Vmin of the voltage signal s1 can be applied across the first wire W1f and the second wire W1s as the largest voltage (also referred to as a maximum voltage).

In the amplifier circuit 22a according to the first embodiment, a maximum value (also referred to as a first maximum current value) Imax1 of a current flowing through the first transistor Tr1 is expressed by Equation 10, for example.

Imax1=V/R=(Vo+|Vmin|)/{(1/gm1)+R1+R3} (Equation 10)

A maximum value (also referred to as a second maximum current value) Imax2 of a current flowing through the second transistor Tr2 is expressed by Equation 11, for example.

Imax2=V/R=(Vo+|Vmin|)/{(1/gm2)+(1/2πfC1)} (Equation 11)

If the reference potential Vo is +10 V, the minimum potential Vmin of the voltage signal s1 is −100 V, the mutual conductance gm1 is 30 mS, the electrical resistance R1 is 1000Ω, and the electrical resistance R3 is 50Ω, for example, the first maximum current value Imax1 calculated from Equation 10 is herein approximately 110 mA. If the mutual conductance gm2 is 133 mS, the frequency f of the alternating current flowing through the first capacitive element Ec1 is 60 MHz, and the capacitance C1 of the first capacitive element Ec1 is 75 pF, for example, the second maximum current value Imax2 calculated from Equation 11 is approximately 2.5 A.

As shown by Equation 1 and Equation 2 above, the voltage Vgs across the gate and the source of each of the first transistor Tr1 and the second transistor Tr2 has a value in accordance with the current I between the source and the drain. In the first embodiment, (W/L) is only required to be set in the first transistor Tr1 so that a variation ΔVgs in voltage

Vgs varied by a flow of a current having the first maximum current value Imax1 is less than a voltage (also referred to as an absolute maximum rating) to cause electrical breakdown of the gate oxide film of the first transistor Tr1, for example. In the first transistor Tr1, the numerical value β is determined in accordance with a manufacturing process of the MOS transistor, for example. Specifically, if the mobility μ is 5.4×10⁻² m²/(V·s) in the first transistor Tr1, for example, a relationship “(variation ΔVgs)<(absolute maximum rating)” can be satisfied when the dielectric constant cox of the oxide contained in the gate oxide film is 3.45×10⁻¹¹ F/m, the thickness tox of the gate oxide film is 7.8×10⁻⁹ m, the channel width W is 500 μm, and the channel length L is 0.35 μm. In the first embodiment, (W/L) is only required to be set in the second transistor Tr2 so that the variation ΔVgs in voltage Vgs varied by a flow of a current having the second maximum current value Imax2 is less than a voltage (an absolute maximum rating) to cause electrical breakdown of the gate oxide film of the second transistor Tr2, for example. If the mobility μ is 2.7×10⁻² m²/(V·s) in the second transistor Tr2, for example, a relationship “(variation ΔVgs)<(absolute maximum rating)” can be satisfied when the dielectric constant εox of the oxide contained in the gate oxide film is 3.45×10⁻¹¹ F/m, the thickness tox of the gate oxide film is 7.8×10⁻⁹ m, the channel width W is 1500 μm, and the channel length L is 0.35 μm.

As described above, in the first embodiment, the variation ΔVgs in voltage Vgs less than the absolute maximum rating, high mutual conductance gm1, and high mutual conductance gm2 can be achieved by the circuit configuration of the amplifier circuit 22a even if the gate oxide film of each of the first transistor Tr1 and the second transistor Tr2 is thin, for example. The amplifier circuit 22a can thus have a high breakdown voltage and high mutual conductance, for example.

In the second transistor Tr2 according to the first embodiment, the 4C electrode E4c as the source electrode and the 4D electrode E4d as the back gate electrode are electrically connected to each other, for example. In other words, the back gate electrode of the second transistor Tr2 is connected to the 4C electrode E4c as the source electrode without being directly connected to the second wire W1s, for example. A malfunction of the second transistor Tr2 is thus less likely to occur when the voltage signal s1 exhibiting the minimum potential Vmin having a large absolute value to generate ultrasound from the first transducer Ut1 is applied to the first transducer Ut1, for example.

<1-4-2. Low Power Consumption of Amplifier Circuit>

As shown in FIGS. 3 to 5, there are three paths along which a current flows from the first wire W1f to the second wire W1s via the amplifier circuit 22a. The three paths include a first path, a second path, and a third path below.

[First Path] A path from the first wire W1f to the second wire W1s via the first transistor Tr1, the first resistive element Er1, and the third resistive element Er3.

[Second Path] A path from the first wire W1f to the second wire W1s via the second transistor Tr2, the second resistive element Er2, and the third resistive element Er3.

[Third Path] A path from the first wire W1f to the second wire W1s via the second transistor Tr2 and the first capacitive element Ec1.

From among these three paths, a direct current can flow along the first path and the second path. The amplifier circuit 22a is thus direct current coupled (also referred to as DC coupled) to the transceiver 4p via the second wire W1s in the first path and the second path. From among the above-mentioned three paths, a direct current does not flow along the third path, and an alternating current flows along the third path. The amplifier circuit 22a is thus alternating current coupled (also referred to as AC coupled) to the transceiver 4p via the second wire W1s in the third path.

In the reference state of the second transducer Ut2 not receiving ultrasound, the direct current i0 as the sum of the direct current i0a in the first path and the direct current i0b in the second path flows from the first wire W1f to the second wire W 1s via the amplifier circuit 22a, for example, as shown in FIG. 4. In a state of the second transducer Ut2 receiving ultrasound, the amplifier circuit 22a has a relationship in which the current i2 in the second path is obtained by amplifying the current i1 in the first path by a small amplification factor (R1/R2), and the alternating current i3 in the third path is obtained by amplifying the current i1 in the first path by a large amplification factor (R1×jωC1), for example, as shown in FIG. 5. The amplifier circuit 22a thus mainly has a function to amplify the alternating current and output the amplified alternating current. The direct current i0 steadily flowing in the amplifier circuit 22a can thus be small, for example. As a result, power consumed in accordance with the direct current i0 steadily flowing in the amplifier circuit 22a can also be small. Power consumption can thereby be reduced in the ultrasound detection device 100, for example.

### 1-5. Summary of First Embodiment

In the ultrasound detection device 100 according to the first embodiment, there are no dedicated wires for grounding and no power supply wires for driving of the switch circuit between the transceiver 4p and the probe portion 22, and there are two wires including the first wire W1f and the second wire W1s therebetween, for example. Even if such a configuration is used, the first transducer Ut1 can transmit ultrasound in response to application of the voltage signal, the second transducer Ut2 can output the electrical signal in response to reception of ultrasound, and the amplifier circuit 22a can output the current signal amplified in response to the electrical signal, for example. The number of wires between the transceiver 4p and the probe portion 22 can thereby be reduced in the ultrasound detection device 100, for example.

### 2. Other Embodiments

The present disclosure is not limited to the above-mentioned first embodiment, and can be modified and improved in various manners without departing from the scope of the present disclosure.

### 2-1. Second Embodiment

In the above-mentioned first embodiment, from among the third resistive element Er3 and the second capacitive element Ec2, the second capacitive element Ec2 may be omitted, for example.

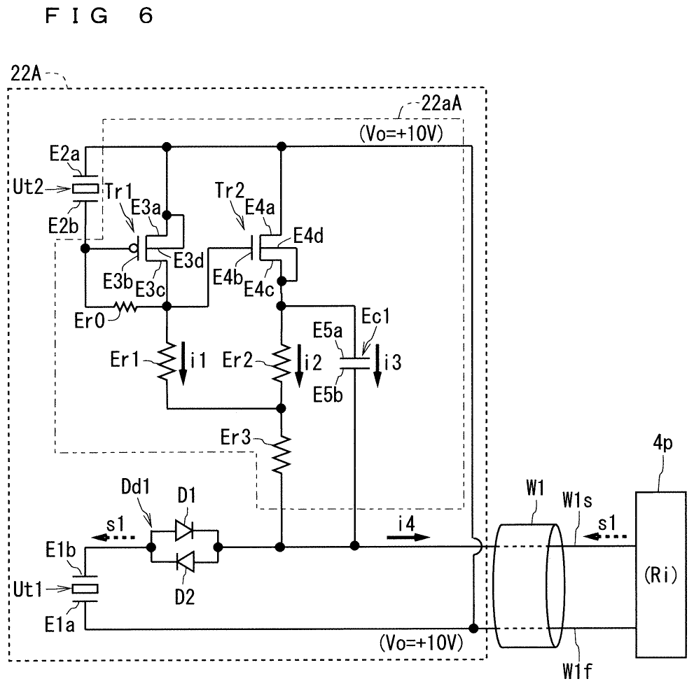

For example, a probe portion 22A shown in FIG. 6 may be used in place of the probe portion 22 according to the first embodiment shown in FIGS. 3 to 5. The probe portion 22A has the configuration of the probe portion 22 according to the first embodiment shown in FIGS. 3 to 5 as a basic configuration, and includes, in place of the amplifier circuit 22a, an amplifier circuit 22aA obtained by omitting the second capacitive element Ec2 from the amplifier circuit 22a. Also in this case, the third resistive element Er3 can cut the high-frequency component of the voltage signal s1 input from the transceiver 4p into the amplifier circuit 22aA via the second wire W1s for application to the first transistor Tr1 and the second transistor Tr2, for example. The change in voltage in the voltage signal s1 applied to the amplifier circuit 22aA can thereby be slowed, for example. As a result, a high breakdown voltage of the amplifier circuit 22aA for application of the voltage signal s1 can be improved, for example. In this case, the amplifier circuit 22aA can easily be implemented on a single semiconductor chip with miniaturization technology, for example.

### 2-2. Third Embodiment

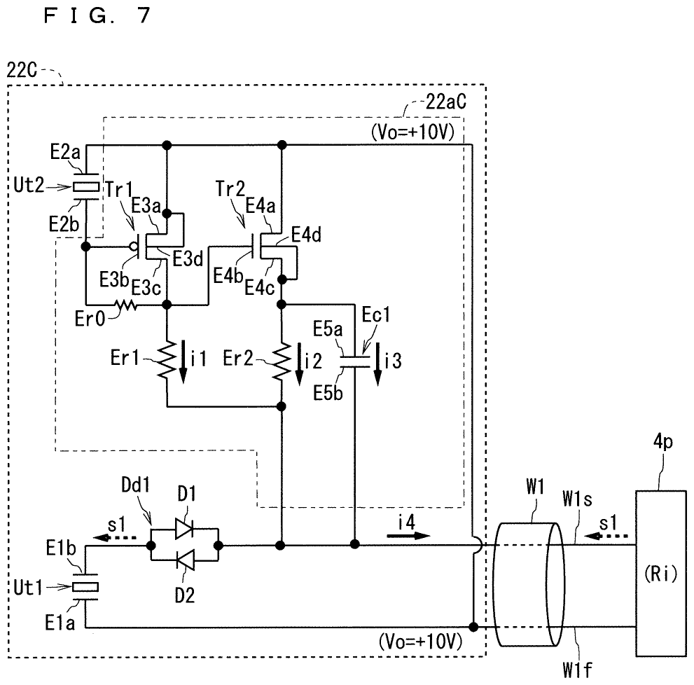

In the above-mentioned first embodiment, both the third resistive element Er3 and the second capacitive element Ec2 may be omitted, for example. For example, a probe portion 22C shown in FIG. 7 may be used in place of the probe portion 22 according to the first embodiment shown in FIGS. 3 to 5. The probe portion 22C has the configuration of the probe portion 22 according to the first embodiment shown in FIGS. 3 to 5 as the basic configuration, and includes, in place of the amplifier circuit 22a, an amplifier circuit 22aC obtained by omitting the third resistive element Er3 and the second capacitive element Ec2 from the amplifier circuit 22a. Also in this case, the number of wires between the transceiver 4p and the probe portion 22C can be reduced, for example.

### 2-3. Fourth Embodiment

In each of the above-mentioned embodiments, the polarity as a whole may be reversed, for example.

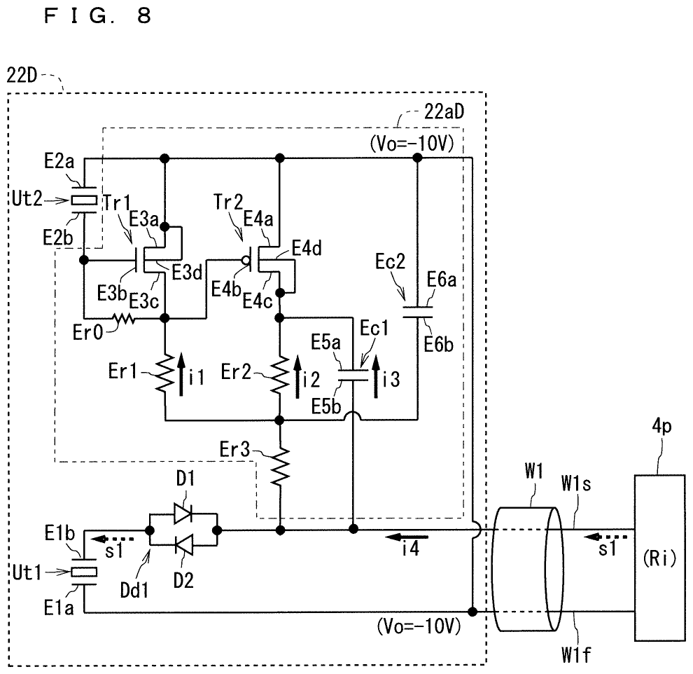

For example, a probe portion 22D shown in FIG. 8 may be used in place of the probe portion 22 according to the first embodiment shown in FIGS. 3 to 5. In this case, the reference potential Vo applied to the probe portion 22D via the first wire W1f is a predetermined negative potential, and the voltage signal s1 applied to the probe portion 22D via the second wire W1s is a pulse exhibiting a positive maximum potential Vmax having a large absolute value, for example. The reference potential Vo is herein set to −10 V or the like, for example. The reference potential Vo may be a predetermined negative potential of approximately −1 V to −30 V, for example. The maximum potential Vmax is set to +100 V or the like, for example.

The probe portion 22D herein has the configuration of the probe portion 22 according to the first embodiment shown in FIGS. 3 to 5 as the basic configuration, and includes an amplifier circuit 22aD shown in FIG. 8 in place of the amplifier circuit 22a. The amplifier circuit 22aD is an amplifier circuit which has the configuration of the amplifier circuit 22a according to the first embodiment shown in FIGS. 3 to 5 as the basic configuration, and in which the first transistor Tr1 has been replaced by an NMOS transistor as the first MOS transistor, and the second transistor Tr2 has been replaced by a PMOS transistor as the second MOS transistor, for example. The NMOS transistor is herein a transistor of the second conductivity type being in the common source configuration and having electrons as majority carriers. The PMOS transistor is a transistor of the first conductivity type being in the common drain configuration and having holes as majority carriers. The first transistor Tr1 herein also includes the 3A electrode E3a as the third electrode acting as the source electrode, the 3B electrode E3b as the fourth electrode acting as the gate electrode, the 3C electrode E3c as the fifth electrode acting as the drain electrode, and the 3D electrode E3d as the back gate electrode electrically connected to the 3A electrode E3a. The second transistor Tr2 includes the 4A electrode E4a as the sixth electrode acting as the drain electrode, the 4B electrode E4b as the seventh electrode acting as the gate electrode, the 4C electrode E4c as the eighth electrode acting as the source electrode, and the 4D electrode E4d as the back gate electrode electrically connected to the 4C electrode E4c, for example.

In the ultrasound detection device 100 according to the fourth embodiment, the currents i1, i2, and i3 flowing through the amplifier circuit 22aD and the signal current i4 flowing through the second wire W1s are opposite in direction or polarity to the currents i1, i2, and i3 flowing through the amplifier circuit 22a and the signal current i4 flowing through the second wire W1s in the abovementioned first embodiment.

Even if such a configuration is used, the first transducer Ut1 can transmit ultrasound in response to application of the voltage signal, the second transducer Ut2 can output the electrical signal in response to reception of ultrasound, and the amplifier circuit 22aD can output the current signal amplified in response to the electrical signal, for example. The number of wires between the transceiver 4p and the probe portion 22 can thereby be reduced in the ultrasound detection device 100, for example.

As in the second embodiment and the third embodiment, the third resistive element Er3 and/or the second capacitive element Ec2 may be omitted herein, for example.

### 2-4. Fifth Embodiment>

In each of the above-mentioned embodiments, the second transistor Tr2 may be replaced by a MOS transistor of the same conductivity type as the first transistor Tr1 in the common source configuration, for example.

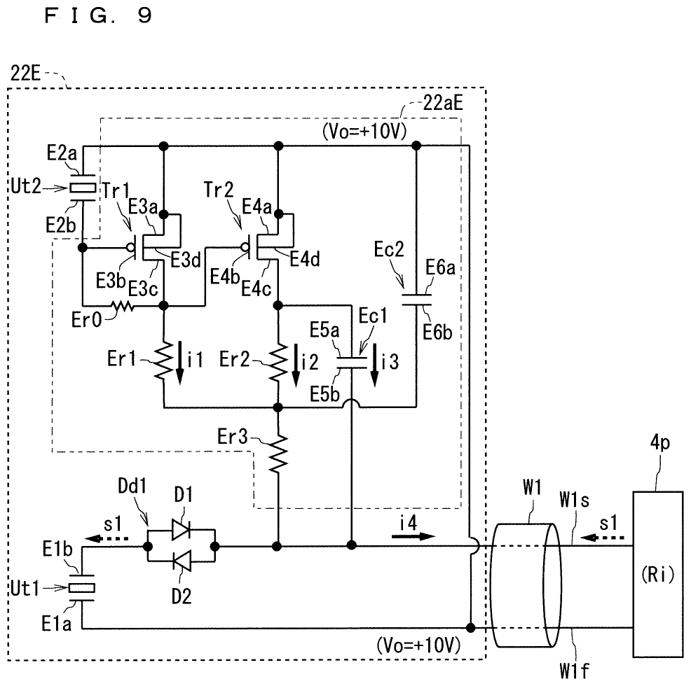

For example, a probe portion 22E shown in FIG. 9 may be used in place of the probe portion 22 according to the first embodiment shown in FIGS. 3 to 5. The probe portion 22E has the configuration of the probe portion 22 according to the first embodiment shown in FIGS. 3 to 5 as the basic configuration, and includes an amplifier circuit 22aE in place of the amplifier circuit 22a, for example. The amplifier circuit 22aE is an amplifier circuit in which the second transistor Tr2 of the amplifier circuit 22a according to the first embodiment has been replaced by a PMOS transistor as the second MOS transistor of the first conductivity type being in the common source configuration and having holes as majority carriers. In this case, the second transistor Tr2 includes the 4A electrode E4a as the sixth electrode acting as the source electrode, the 4B electrode E4b as the seventh electrode acting as the gate electrode, and the 4C electrode E4c as the eighth electrode acting as the drain electrode. The 4A electrode E4a is connected to the first wire W1f. The 4B electrode E4b is electrically connected to the 3C electrode E3c of the first transistor Tr1. The second transistor Tr2 also includes the 4D electrode E4d as the back gate electrode electrically connected to the 4A electrode E4a, for example. The electrical resistance R2 of the second resistive element Er2 may herein be reduced relative to the electrical resistance R1 of the first resistive element Er1, for example. In this case, the amplification factor to amplify the voltage V1 into the voltage V2 can be reduced to increase the band ωc2 of the signal strength enabling amplification of the voltage V1 into the voltage V2, for example. As a result, the capacitance C1 as a drive capability of the first capacitive element Ec1 can be increased, for example.

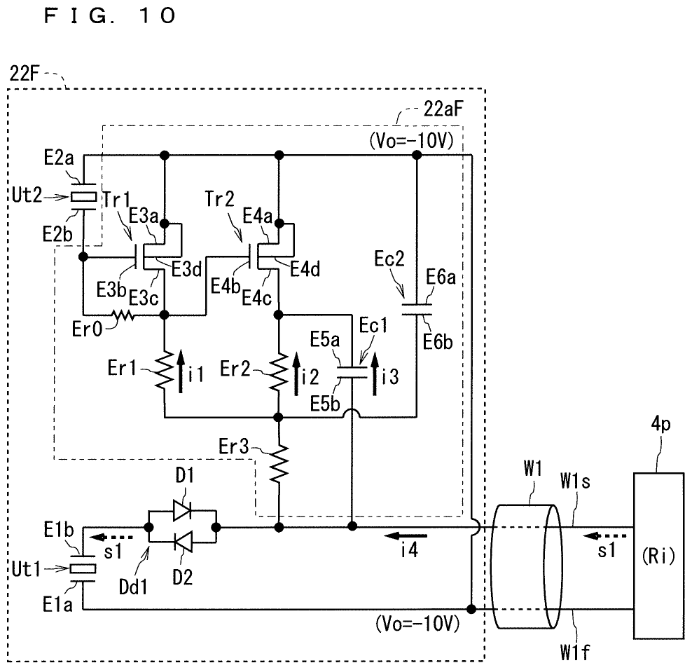

For example, a probe portion 22F shown in FIG. 10 may be used in place of the probe portion 22D according to the fourth embodiment shown in FIG. 8. The probe portion 22F has the configuration of the probe portion 22D according to the fourth embodiment shown in FIG. 8 as the basic configuration, and includes an amplifier circuit 22aF in place of the amplifier circuit 22aD. The amplifier circuit 22aF is an amplifier circuit in which the second transistor Tr2 of the amplifier circuit 22aD according to the fourth embodiment has been replaced by an NMOS transistor as the second MOS transistor of the second conductivity type being in the common source configuration and having electrons as majority carriers. In this case, the second transistor Tr2 includes the 4A electrode E4a as the sixth electrode acting as the source electrode, the 4B electrode E4b as the seventh electrode acting as the gate electrode, and the 4C electrode E4c as the eighth electrode acting as the drain electrode. The 4A electrode E4a is connected to the first wire W1f. The 4B electrode E4b is electrically connected to the 3C electrode E3c of the first transistor Tr1. The second transistor Tr2 also includes the 4D electrode E4d as the back gate electrode electrically connected to the 4A electrode E4a.

### 2-5. Sixth Embodiment

In each of the above-mentioned embodiments, the MOS transistors as the first transistor Tr1 and the second transistor Tr2 may be bipolar transistors, for example. If such a configuration is used, the bipolar transistors each do not include the gate oxide film in which the electrical breakdown can occur in contrast to the MOS transistors, for example. Problems of the first transistor Trl and the second transistor Tr2 are thus less likely to occur when the voltage signal sl exhibiting the minimum potential Vmin or the maximum potential Vmax having a large absolute value to generate ultrasound from the first transducer Ut1 is applied to the first transducer Ut1, for example.

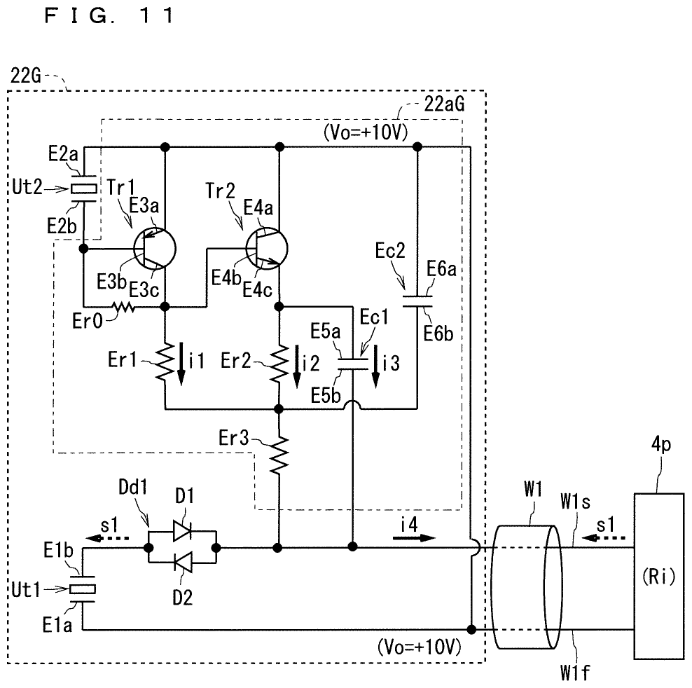

In this case, a probe portion 22G shown in FIG. 11 may be used in place of the probe portion 22 according to the first embodiment shown in FIGS. 3 to 5, for example. The probe portion 22G has the configuration of the probe portion 22 according to the first embodiment shown in FIGS. 3 to 5 as the basic configuration, and includes an amplifier circuit 22aG in place of the amplifier circuit 22a, for example. The amplifier circuit 22aG is an amplifier circuit which has the configuration of the amplifier circuit 22a according to the first embodiment as the basic configuration, and in which the MOS transistors as the first transistor Tr1 and the second transistor Tr2 have been replaced by the bipolar transistors. The first transistor Tr1 is herein a PNP-type transistor as a transistor of the first conductivity type in a common emitter configuration, for example. The second transistor Tr2 is an NPN-type transistor as a transistor of the second conductivity type in a common collector configuration, for example. In this case, the first transistor Tr1 includes the 3A electrode E3a as the third electrode acting as an emitter electrode, the 3B electrode E3b as the fourth electrode acting as a base electrode, and the 3C electrode E3c as the fifth electrode acting as a collector electrode. The 3A electrode E3a is electrically connected to the first wire W1f. The 3B electrode E3b is electrically connected to the 2B electrode E2b as the second electrode of the second transducer Ut2. The 3C electrode E3c is electrically connected to the 3B electrode E3b via the resistive element Er0 to form the diode connection. The second transistor Tr2 includes the 4A electrode E4a as the sixth electrode, the 4B electrode E4b as the seventh electrode acting as the base electrode, and the 4C electrode E4c as the eighth electrode. The 4A electrode E4a can herein act as the collector electrode, for example. The 4C electrode E4c can act as the emitter electrode, for example. The 4A electrode E4a is electrically connected to the first wire W1f. The 4B electrode E4b is electrically connected to the 3C electrode E3c of the first transistor Tr1.

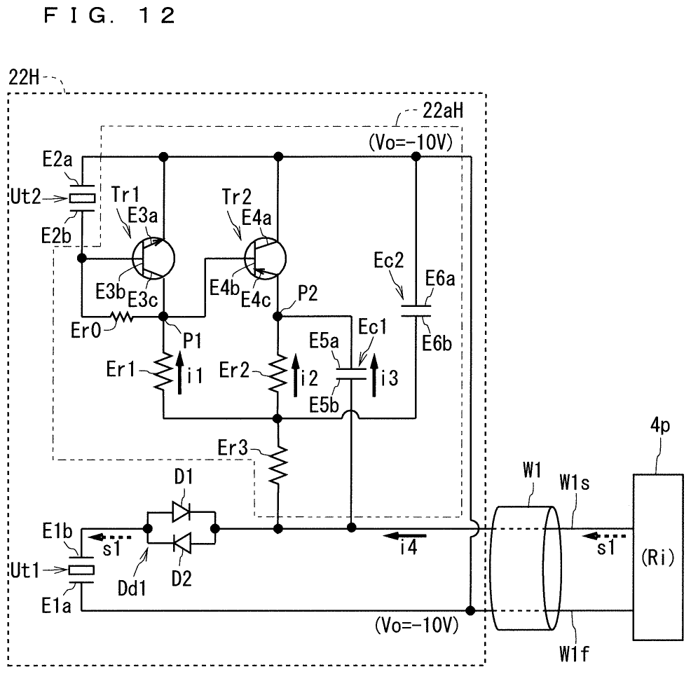

For example, a probe portion 22H shown in FIG. 12 may be used in place of the probe portion 22D according to the fourth embodiment shown in FIG. 8. The probe portion 22H has the configuration of the probe portion 22D according to the fourth embodiment shown in FIG. 8 as the basic configuration, and includes an amplifier circuit 22aH in place of the amplifier circuit 22aD, for example. The amplifier circuit 22aH is an amplifier circuit which has the configuration of the amplifier circuit 22aD according to the fourth embodiment as the basic configuration, and in which the MOS transistors as the first transistor Tr1 and the second transistor Tr2 have been replaced by the bipolar transistors. The first transistor Tr1 is herein the NPN-type transistor as a transistor of the second conductivity type in the common emitter configuration, for example. The second transistor Tr2 is the PNP-type transistor as a transistor of the first conductivity type in the common collector configuration, for example. In this case, the first transistor Tr1 includes the 3A electrode E3a as the third electrode acting as the emitter electrode, the 3B electrode E3b as the fourth electrode acting as the base electrode, and the 3C electrode E3c as the fifth electrode acting as the collector electrode. The 3A electrode E3a is electrically connected to the first wire W1f. The 3B electrode E3b is electrically connected to the 2B electrode E2b as the second electrode of the second transducer Ut2. The 3C electrode E3c is electrically connected to the 3B electrode E3b via the resistive element Er0 to form the diode connection. The second transistor Tr2 includes the 4A electrode E4a as the sixth electrode, the 4B electrode E4b as the seventh electrode acting as the base electrode, and the 4C electrode E4c as the eighth electrode. The 4A electrode E4a can herein act as the collector electrode. The 4C electrode E4c can act as the emitter electrode, for example. The 4A electrode E4a is electrically connected to the first wire W1f. The 4B electrode E4b is electrically connected to the 3C electrode E3c of the first transistor Tr1.

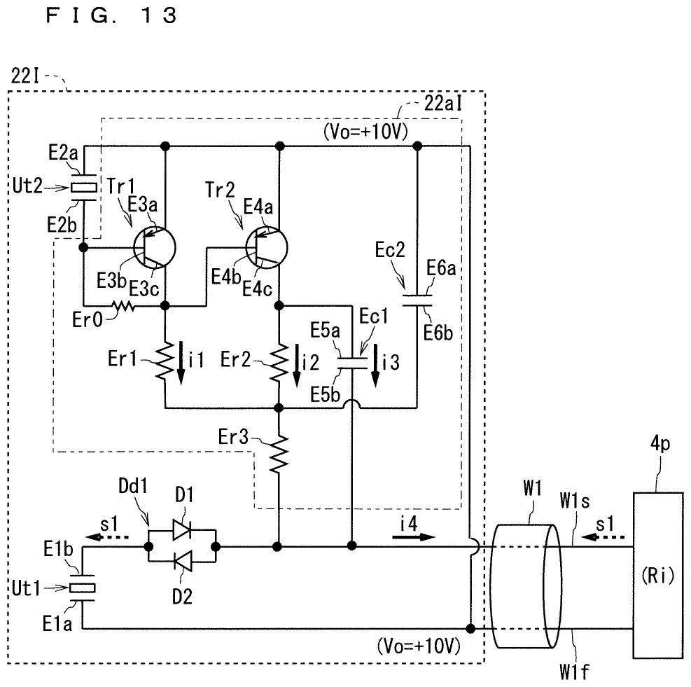

For example, a probe portion 22I shown in FIG. 13 may be used in place of the probe portion 22E according to the fifth embodiment shown in FIG. 9. The probe portion 221 has the configuration of the probe portion 22E according to the fifth embodiment shown in FIG. 9 as the basic configuration, and includes an amplifier circuit 22aI in place of the amplifier circuit 22aE, for example. The amplifier circuit 22aI is an amplifier circuit which has the configuration of the amplifier circuit 22aE according to the fifth embodiment as the basic configuration, and in which the MOS transistors as the first transistor Tr1 and the second transistor Tr2 have been replaced by the bipolar transistors. The first transistor Tr1 and the second transistor Tr2 are herein each the PNP-type transistor as the transistor of the first conductivity type in the common emitter configuration, for example. The first transistor Tr1 includes the 3A electrode E3a as the third electrode acting as the emitter electrode, the 3B electrode E3b as the fourth electrode acting as the base electrode, and the 3C electrode E3c as the fifth electrode acting as the collector electrode. The 3A electrode E3a is electrically connected to the first wire W1f. The 3B electrode E3b is electrically connected to the 2B electrode E2b as the second electrode of the second transducer Ut2. The 3C electrode E3c is electrically connected to the 3B electrode E3b via the resistive element Er0 to form the diode connection. The second transistor Tr2 includes the 4A electrode E4a as the sixth electrode, the 4B electrode E4b as the seventh electrode acting as the base electrode, and the 4C electrode E4c as the eighth electrode. The 4A electrode E4a can herein act as the emitter electrode, for example. The 4C electrode E4c can act as the collector electrode, for example. The 4A electrode E4a is electrically connected to the first wire W1f. The 4B electrode E4b is electrically connected to the 3C electrode E3c of the first transistor Tr1.

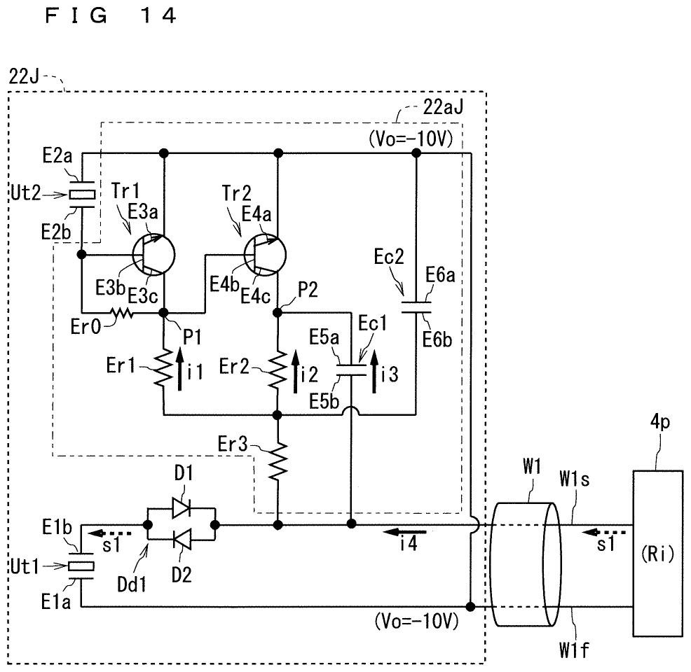

For example, a probe portion 22J shown in FIG. 14 may be used in place of the probe portion 22F according to the fifth embodiment shown in FIG. 10. The probe portion 22J has the configuration of the probe portion 22F according to the fifth embodiment shown in FIG. 10 as the basic configuration, and includes an amplifier circuit 22aJ in place of the amplifier circuit 22aF, for example. The amplifier circuit 22aJ is an amplifier circuit which has the configuration of the amplifier circuit 22aF according to the fifth embodiment as the basic configuration, and in which the MOS transistors as the first transistor Tr1 and the second transistor Tr2 have been replaced by the bipolar transistors. The first transistor Tr1 and the second transistor Tr2 are herein each the NPN-type transistor as the transistor of the second conductivity type in the common emitter configuration, for example. In this case, the first transistor Tr1 includes the 3A electrode E3a as the third electrode acting as the emitter electrode, the 3B electrode E3b as the fourth electrode acting as the base electrode, and the 3C electrode E3c as the fifth electrode acting as the collector electrode. The 3A electrode E3a is electrically connected to the first wire W1f. The 3B electrode E3b is electrically connected to the 2B electrode E2b as the second electrode of the second transducer Ut2. The 3C electrode E3c is electrically connected to the 3B electrode E3b via the resistive element Er0 to form the diode connection. The second transistor Tr2 includes the 4A electrode E4a as the sixth electrode, the 4B electrode E4b as the seventh electrode acting as the base electrode, and the 4C electrode E4c as the eighth electrode. The 4A electrode E4a can herein act as the emitter electrode, for example. The 4C electrode E4c can act as the collector electrode, for example. The 4A electrode E4a is electrically connected to the first wire W1f. The 4B electrode E4b is electrically connected to the 3C electrode E3c of the first transistor Tr1.

### 2-6. Others

The ultrasound detection device 100 according to each of the above-mentioned embodiments may be used in applications other than applications where the tomographic image in the blood vessel is to be viewed, such as applications where a tomographic image of a surrounding object in a pipe or a narrow space is to be viewed. In this case, the ultrasound detection device 100 is only required to include the elongated sensor portion 21 and the transceiver 4p, for example.

It is needless to say that all or some of the above-mentioned embodiments and various modifications can appropriately be combined unless any contradictions occur.

1. An ultrasound detection device comprising:
a probe portion capable of transmitting and receiving ultrasound to and from an object;
a transceiver capable of transmitting and receiving a signal to and from the probe portion, applying a power supply voltage to the probe portion, and detecting a voltage in accordance with a current signal using an internal resistor;
a first wire electrically connecting the transceiver and the probe portion, and enabling the transceiver to apply a reference potential to the probe portion; and
a second wire electrically connecting the transceiver and the probe portion, and enabling the transceiver to transmit a voltage signal to the probe portion and enabling the probe portion to transmit the current signal to the transceiver, wherein
the probe portion includes:
a first transducer capable of transmitting ultrasound to the object in response to application of the voltage signal;
a second transducer capable of receiving ultrasound from the object, and converting the ultrasound into an electrical signal, the second transducer including a first electrode electrically connected to the first wire and a second electrode;
a bidirectional diode connecting the second wire and the first transducer; and
an amplifier circuit capable of amplifying a current, and outputting a current signal to the second wire in response to the electrical signal,
further wherein the amplifier circuit includes:
a first transistor including a third electrode connected to the first wire and acting as a source or an emitter, a fourth electrode electrically connected to the second electrode and acting as a gate or a base, and a fifth electrode electrically connected to the fourth electrode via a resistive element and acting as a drain or a collector, the first transistor being in a common source configuration or a common emitter configuration;
a first resistive element electrically connecting the fifth electrode and the second wire;
a second transistor including a sixth electrode connected to the first wire, a seventh electrode electrically connected to the fifth electrode and acting as a gate or a base, and an eighth electrode, the second transistor being of a different conductivity type from the first transistor and being in a common drain configuration or a common collector configuration, or being of the same conductivity type as the first transistor and being in a common source configuration or a common emitter configuration;
a second resistive element electrically connecting the eighth electrode and the second wire; and
a first capacitive element connecting the eighth electrode and the second wire.

2. The ultrasound detection device according to claim 1, wherein
the bidirectional diode allows the voltage signal to pass and does not allow a signal having a strength less than a threshold to pass from the second wire to the first transducer.

3. The ultrasound detection device according to claim 1, wherein
the first capacitive element includes a ninth electrode electrically connected to the eighth electrode and a tenth electrode electrically connected to the second wire.

4. The ultrasound detection device according to claim 1, wherein
the first transistor is of a first conductivity type,
the sixth electrode includes a drain or a collector,
the eighth electrode includes a source or an emitter, and
the second transistor includes a transistor of a second conductivity type in the common drain configuration or the common collector configuration.

5. The ultrasound detection device according to claim 1, wherein
the first transistor is of a second conductivity type,
the sixth electrode includes a drain or a collector,
the eighth electrode includes a source or an emitter, and
the second transistor includes a transistor of a first conductivity type in the common drain configuration or the common collector configuration.

6. The ultrasound detection device according to claim 1, wherein
the first transistor is of a first conductivity type,
the sixth electrode includes a source or an emitter,
the eighth electrode includes a drain or a collector, and
the second transistor includes a transistor of the first conductivity type in the common source configuration or the common emitter configuration.

7. The ultrasound detection device according to claim 1, wherein
the first transistor is of a second conductivity type,
the sixth electrode includes a source or an emitter,
the eighth electrode includes a drain or a collector, and
the second transistor includes a transistor of the second conductivity type in the common source configuration or the common emitter configuration.

8. The ultrasound detection device according to claim 4, wherein
the third electrode includes the source,
the fourth electrode includes the gate,
the fifth electrode includes the drain,
the first transistor includes a first MOS transistor of the first conductivity type,
the sixth electrode includes the drain,
the seventh electrode includes the gate,
the eighth electrode includes the source, and
the second transistor includes a second MOS transistor of the second conductivity type.

9. The ultrasound detection device according to claim 5, wherein
the third electrode includes the source,
the fourth electrode includes the gate,
the fifth electrode includes the drain,
the first transistor includes a first MOS transistor of the second conductivity type,
the sixth electrode includes the drain,
the seventh electrode includes the gate,
the eighth electrode includes the source, and
the second transistor includes a second MOS transistor of the first conductivity type.

10. The ultrasound detection device according to claim 6, wherein
the third electrode includes the source,
the fourth electrode includes the gate,
the fifth electrode includes the drain,
the first transistor includes a first MOS transistor of the first conductivity type,
the sixth electrode includes the source,
the seventh electrode includes the gate,
the eighth electrode includes the drain, and
the second transistor includes a second MOS transistor of the first conductivity type.

11. The ultrasound detection device according to claim 7, wherein
the third electrode includes the source,
the fourth electrode includes the gate,
the fifth electrode includes the drain,
the first transistor includes a first MOS transistor of the second conductivity type,
the sixth electrode includes the source,
the seventh electrode includes the gate,
the eighth electrode includes the drain, and
the second transistor includes a second MOS transistor of the second conductivity type.

12. The ultrasound detection device according to claim 8, wherein
the second MOS transistor includes a back gate electrode electrically connected to the eighth electrode.

13. The ultrasound detection device according to claim 1, wherein
the first transistor includes a bipolar transistor in the common emitter configuration, and
the second transistor includes a bipolar transistor of a different conductivity type from the first transistor in the common collector configuration or a bipolar transistor of the same conductivity type as the first transistor in the common emitter configuration.

14. The ultrasound detection device according to claim 1, wherein
the amplifier circuit further includes a third resistive element electrically connecting each of the first resistive element and the second resistive element to the second wire.

15. The ultrasound detection device according to claim 14, wherein
the amplifier circuit further includes a second capacitive element,
the first capacitive element includes a ninth electrode connected to the eighth electrode and a tenth electrode connected to the second wire, and
the second capacitive element includes an eleventh electrode electrically connected to the first wire and a twelfth electrode electrically connected to the second wire via the third resistive element.

16. The ultrasound detection device according to claim 9, wherein
the second MOS transistor includes a back gate electrode electrically connected to the eighth electrode.
